(19) United States
(12) Patent Application Publication
(10) Pub. No.: US 2008/0300227 A1
(43) Pub. Date: Dec. 4, 2008

(54) Drugs for treating hypertension combined with hyperuricemia and/or hypercholesterolemia

(75) Inventors: Akira Tomiyama, Sakaki-machi (JP); Hiroshi Tomiyama, Sakaki-machi (JP); Hidetoshi Miyamoto, Chikuma-shi (JP); Keiichoro Hayashi, Ueda-shi (JP); Seiichiro Mochizuki, Ueda-shi (JP)

Correspondence Address: FLYNN THIEL BOUTELL & TANIS, P.C., 2026 RAMBLING ROAD, KALAMAZOO, MI, 49008-1631, US

(73) Assignee: KOTOBUKI PHARMACEUTICAL CO., LTD.

(21) Appl. No.: 12/221,385

(22) Filed: Aug. 1, 2008

Related U.S. Application Data
(62) Division of application No. 11/629,689, filed on Dec. 14, 2006.

(30) Foreign Application Priority Data
Jun. 23, 2004 (JP) 2004/184906

(51) Int. Cl. A61K 31/56; A61K 31/41; A61K 31/5415; A61P 9/12; A61K 31/498; A61K 31/4965

(52) U.S. Cl. 514/171; 514/381; 514/223.5; 514/249; 514/255.06

(57) ABSTRACT

The invention is directed to a drug for treating hypertension combined with serum hyperuricemia and/or hypercholesterolemia, with the active ingredients being 2-propyl-3-{[21-(1H-tetrazole-5-yl)biphenyl-4-yl]methyl}-5,6,7, 8-tetrahydrocyclohepta imidazole-4-(3H)-one and a prodrug or salt thereof. Pratosartan can be used in combination with one or more diuretics chosen from sulfonamide-, phenoxyacetic acid- and thiazide-type diuretics, triamterene, amiloride, spironolactone, potassium canrenoate and traxanox sodium. Also, pratosartan can be used in combination with one or more hypolipidemic drugs chosen from statins, fibrates, cholesterol absorption inhibitors, cholesterol sequestrants and cholesterol excretion enhancers.

This is a division of Ser. No. 11/629689, filed Dec. 14, 2006, which was the national stage of International Application No. PCT/JP2004/15461, filed Oct. 13, 2004, which International Application was not published in English.

### TECHNICAL FIELD

This invention is related to drugs for treating hypertension combined with hyperuricemia and/or hypercholesterolemia.

### BACKGROUND TECHNOLOGY

Recently, various studies for prevention and therapy of geriatric diseases are performed. Risk factors of geriatric diseases are hypertension, hypercholesterolemia, diabetes mellitus, obesity and hyperuricemia. And, the mortality markedly increases when these risk factors overlap more than two. Also, it is known that among this overlap of the risk factor, the overlap of hypertension and hyperuricemia, of hypertension and hypercholesterolemia are much frequency.

Conventionally, angiotensin II receptor blockers are widely used for therapy of hypertension. And, the inhibitory action on atherosclerosis development of angiotensin II receptor blockers is shown in animal experiments as secondary or indirect action of hypotensive effect. However, there is no report that angiotensin II receptor blocker decreases plasma cholesterol level or LDL level. It is known that 2-propyl-3-{[2′-(1H-tetrazole-5-yl)biphenyl-4-yl]methyl}-5,6,7,8-tetrahydrocyclohepta imidazole -4-(3H)-one (General name : pratosartan) in this invention has angiotensin II receptor blocking action and is effective for therapy of hypertension and cardiac failure (JPB 707390). It is known for a long time that most diuretics, which are generally used same as angiotensin II receptor blockers in clinical situation, itself increase uric acid levels, and it is reported that sulfonamide and thiazide-type diuretics increase serum uric acid levels (Joel G. Hardmann et al., The Pharmacological Basis of Therapeutics, 10th edition, McGraw-Hill (USA), 2001, page 757-787). Also, it is reported that some of angiotensin II receptor blockers increase uric acid levels or have no uric acid excretion action (Saitoh, M. et al., “Uricosuric Effects of Angiotensin II Receptor Antagonist in the Patients with Normal Renal Function”, Japanese Journal of Clinical Pharmacology and Therapeutics, 2003, 34(2), 37-42. “Journal of Hypertensions”, 2001, 19(10), 1855-1860). Nevertheless, conventional co-administration of angiotensin II blocker and diuretics is an important therapeutic method because of certain hypotensive action (International publication No. 89/6233 pamphlet, JPA H3(1991)-27362). In the guideline for hypertension therapy, in regard to uric acid level of hypertension, it is desirable to choose the hypotensive drugs that are not increase uric acid level at least. (Guideline Subcommittee of the Japanese Society of Hypertension, JSH2000 Hypertension Guidelines for General Practitioners, 1st edition, The Japanese Society of Hypertension, Jun. 30, 2000, page 55-58). In the view of this increase of uric acid, there are some problems about conventional co-administration of angiotensin II blocker and diuretics. Under these circumstances, angiotensin II blockers, which are able to decrease uric acid level, are expected, and the dosage of those alone or a combination drug are also expected.

Also, it is known that high dose of thiazide- and loop-type diuretics increase serum cholesterol level, triglyceride and LDL-cholesterol (Guideline Subcommittee of the Japanese Society of Hypertension, JSH2000 Hypertension Guidelines for General Practitioners, 1st edition, The Japanese Society of Hypertension, Jun. 30, 2000, page 55-58). In hypercholesterolemia, sufficient hypocholesterolemic effect is not obtained when one drug is used occasionally, so co-administration by multiple hypocholesterolemic drugs is performed in clinical situation generally.

In this invention, the purpose is to providing drugs for treating hypertension combined with serum hyperuricemia and/or hypercholesterolemia that is much frequency among the duplication onset in geriatric diseases.

### DISCLOSURE OF INVENTION

Inventors of this invention newly discovered that 2-propyl-3-{[2′-(1H-tetrazole -5-yl) biphenyl -4-yl]methyl}-5,6,7, 8-tetrahydrocyclohepta imidazole -4-(3H) - one (General name pratosartan), which is one of the angiotensin II blockers, have hypouricosuric action and hypolipidemic action (hypocholesterolemic action and LDL lowering action) other than hypotensive action by animal studies and clinical trials, and completed this invention.

In other words, this invention is related to drugs for treating hypertension combined with serum hyperuricemia and/or hypercholesterolemia of which the active ingredient is 2-propyl-3-{[2′-(1H-tetrazole -5-yl) biphenyl -4-yl]methyl}-5,6,7, 8-tetrahydrocyclohepta imidazole -4-(3H) - one and a prodrug or salt thereof.

Also, this invention is related to drugs for treating hypertension combined with serum hyperuricemia and/or hypercholesterolemia, of which the active ingredient is 2-propyl-3- {[2′-(1H-tetrazole -5-yl) biphenyl -4-yl]methyl}-5, 6,7,8-tetrahydrocyclohepta imidazole -4-(3H) - one, and a prodrug or salt thereof and a diuretic. As the diuretic, one or more diuretics are preferably chosen from sulfonamide-, phenoxyacetic acid- and thiazide-type diuretics, triamterene, amiloride, spironolactone, potassium canrenoate and traxanox sodium. Also, as a diuretic, thiazide-type diuretics are preferably chosen from the group consisting of hydrochlorothiazide, methyclothiazide, benzylhydrochloro- thiazide, trichloromethiazide, cyclopenthiazide, polythiazide, ethiazide, cyclothiazide, bendroflumethiazide and hydroflumethiazide.

Also, this invention is related to drugs for treating hypertension combined with serum hyperuricemia and/or hypercholesterolemia, of which the active ingredient is 2-propyl-3- {[2′-(1H-tetrazole -5-yl) biphenyl -4-yl]methyl}-5, 6,7,8-tetrahydrocyclohepta imidazole -4-(3H) - one, and a prodrug or salt thereof, and a hypolipidemic drug. As the hypolipidemic drug, one or more diuretics are preferably chosen from the group consisting of statins, fibrates, cholesterol sequestrants, cholesterol absorption inhibitors and cholesterol excretion enhancers.

### BRIEF DESCRIPTION OF DRAWING

FIG. 1 is a graph showing the result of serum total cholesterol measured, and FIG. 2 is a graph showing the result of LDL-cholesterol measured.

### BEST MODE FOR CARRYING OUT THE INVENTION

Pratosartan alone or mixed with appropriate pharmaceutical acceptable excipients or dilutions, for example oral dosage such as tablet, capsule, granule, powder or syrup, or non-oral dosage such as suppository. Though the dose is varied by condition, age and others, in oral dosage, the dose of pratosartan varies from 1 mg of lower limit to 1000 mg of upper limit. The dosage number is from one to five.

Pratosartan has a plasma uric acid level decreasing action and is useful as a plasma uric acid excretion enhancer and uricosuric drug. Also, because of the hypotensive action of pratosartan, it is expected to ameliorate hypertension with hypouricemia, and is useful for therapy of hyperuricemia with hypertension. Furthermore, pratosartan has hypocholesterolemic action and is useful as a hypolipidemic drug of plasma cholesterol.

Also, because of the hypotensive action of pratosartan, it is expected to ameliorate hypertension with hypolipidemia or atherosclerosis, and is useful for therapy of hypolipidemia or atherosclerosis with hypertension.

Also, pratosartan is able to be used in combination with a diuretic. Though diuretics, especially sulfonamide- and thiazide-type diuretics, have a hypotensive action, they also have an adverse effect of increasing the plasma uric acid level. On the other hand, many conventional angiotensin II receptor blockers increase the plasma uric acid level. Therefore, it is difficult to administer diuretics with conventional angiotensin II receptor blockers. However, pratosartan has an advantage of decreasing the plasma uric acid level, so pratosartan co-administered with a diuretic show the hypotensive action of a diuretic with the inhibition of increasing the plasma uric acid level and, because pratosartan itself shows a hypotensive action, synergistic hypotensive actions are obtained by co-administration with a diuretic. Co-administration of pratosartan with a diuretic is expected as a therapeutic drug for hypertension, hypertension with hyperuricemia and cardiovascular diseases. Also, though it is known that a high dose of thiazide- and loop-type diuretics increase the serum total cholesterol, triglyceride and LDL cholesterol (Guideline Subcommittee of the Japanese Society of Hypertension, JSH2000 Hypertension Guidelines for General Practitioners, 1st edition, The Japanese Society of Hypertension, Jun. 30, 2000, page 55-58), the hypocholesterolemic action of pratosartan reduces this hindrance.

The diuretics used are sulfonamide-, phenoxyacetic acid- and thiazide-type diuretics and others. Also, among these diuretics, especially sulfonamide- and thiazide-type diuretics have an adverse effect of increasing the serum uric acid level, but co-administration of pratosartan reduces the increase of the uric acid level, which is an adverse effect of these diuretics.

The above sulfonamide-type diuretics are acetazolamide, metazolamide, ethoksuzolamide, clofenamide, dichlorfenamide, disulfamide, mefluside, chlorthalidone, kinetazone, furosemide, clopamide, tripamide, indapamide, crolexolone, metrazone, xipamide, bumetanide, piretanide and others. Also, the above thiazide-type diuretics are hydrochlorthiazide, methiclothiazide, benzylchlorthiazide, trichlormethiazide, cyclopenthiazide, polythiazide, ethiazide, cyclothiazide,bendroflumethiazide, hydroflumethiazide and others. Also, ethacrynic acid, thienilic acid, quincarbate, indaclinone and others. The other diuretics are triamterene, amiloride, spironolactone, potassium canrenoate and traxanox sodium. Suitable diuretics are thiazide-type diuretics, and hydrochlorthiazide is more suitable.

In this invention, in the case of co-administration of pratosartan and diuretics, the ratio of pratosartan and diuretics markedly varies, the ratio of mass is appropriate from 1:500 to 500:1. Both low dose of pratosartan and diuretics, which does not produce adverse effects, are able to lower the blood pressure synergistically.

Also, the co-administration of pratosartan and conventional. hypolipidemic drugs is able to reinforce the hypolipidemic action. Because pratosartan itself has a hypotensive action, it is expected that the co-administartion of pratosartan and diuretics show a therapy for geriatric diseases such as hypertension and hyperlipidemia, and cardiovascular disorder induced by the combination of the above-mentioned diseases. These conventional hypolipidemic drugs are at least one sort chosen from the group consisting of statins, fibrates, cholesterol absorption inhibitors, cholesterol sequestrants and cholesterol excretion enhancers. The above statins are atorvastatin, simvastatin, pravastatin, fluvastatin, pitavastatin and others. Fibrates are clinofibrate, clofibrate, simfibrate, fenofibrate, bezafibrate and others. Cholesterol absorption inhibitors are ezetimibe, soysterol and others. Cholesterol sequestrants are cholestimide, cholestyramine and others. Cholesterol excretion enhancers are probucol and others. In the case of co-administration of pratosartan and conventional hypolipidemic drugs, the ratio of pratosartan and conventional hypolipidemic drugs markedly varies, the ratio of mass is appropriate from 1:500 to 500:1. Both low doses of pratosartan and conventional hypolipidemic drugs, which does not produce adverse effects, are able to lower the serum lipid level, especially LDL level, to the target value of therapy shown in The Guideline of Diagnosis and Treatment of Hyperlipidemia (Japan Atherosclerosis Society, 1997).

Working examples (experimental examples) are shown for the explanation of the invention in more detail as follows, but these examples do not limit this invention.

### EXAMPLE 1

The uricosuric action is estimated by the delayed disappearance of blood phenol red level as a marker. Namely, male 6 weeks old SD rats were orally administered 0.5% methylcellulose (MC) or pratosartan (30 mg/kg or 100 mg/kg) and, after three hours, phenol red solution (75 mg/kg) was intravenously administered. One hour after phenol red administration, blood samples were collected from abdominal aorta under ether anesthesia, and the amount of phenol red in the serum was determined. The amount of phenol red level in the control group was taken as 100% and the delayed rate of phenol red disappearance from the blood in the pratosartan treated group was estimated as the enhanced rate of uric acid excretion (%). In Table 1 are experimental groups, subject substances, doses and the enhanced rates of uric acid excretion (mean±standard deviation). As shown in Table 1, pratosartan enhances the excretion of uric acid.

TABLE 1

| | Subject substances | The enhance rate of uric acid |
|---|---|---|
| Groups | and doses | excretion (%) |
| Group 1 | 0.5% MC 2 mL/kg | 100 ± 5 |
| Group 2 | pratosartan 30 mg/kg | 105 ± 10 |
| Group 3 | pratosartan 100 mg/kg | 172 ± 17 |

### EXAMPLE 2

Studies were carried out intended for mild and moderate essential hypertension. 17 patients afflicted with hyperuricemia were administered pratosartan and the serum uric acid levels (mg/dL) before drug administration and the last day during administration were determined. The results are shown in Table 2. Further, a dose of from 40 to 160 mg/day was administered every four weeks by degrees if the hypotensive action was insufficient.

TABLE 2

| | Serum uric acid level (mg/dL) | | |
|---|---|---|---|
| No. of | Before drug | The last day during | |
| patients | administration | administration | Difference |
| 17 | 8.0 ± 1.1 | 7.4 ± 1.0 | −0.7 ± 1.3 |

### EXAMPLE 3

The systolic blood pressure levels of male 20 weeks old SHR (Spontaneously hypertensive rat, SPF grade) were measured by tail cuff methods and the animals were randomized into 4 groups to balance the systolic blood pressure level among groups. 0.5% methylcellulose (MC) or drugs suspended in 0.5% MC were administered for 28 days and the systolic blood pressure levels after 5 hours of drug administration were measured at day 1, 7, 14 and 28. Table 3 shows the experimental groups, subject substances and doses and the measured blood pressure levels (mean ±standard deviation) are shown in Table 4. Further, the subject substances were hydrochlorothiazide (HCTZ), pratosartan, and co-administration of HCTZ and pratosartan. The dose of HCTZ and pratosartan were 10 mg/kg and 3 mg/kg, respectively, and the dosage volume was 2 mL/kg in each case. The hypotensive action synergistically enhanced that of HCTZ.

TABLE 3

| | |
|---|---|
| Groups | Subject substances and doses |

| | | | |
|---|---|---|---|
| Group 1 | 0.5% MC | 2 | mL/kg |
| Group 2 | HCTZ | 10 | mg/kg |
| Group 3 | Pratosartan | 3 | mg/kg |
| Group 4 | HCTZ 10 mg/kg + pratosartan | 3 | mg/kg |

TABLE 4

| | Group 1 | Group 2 | Group 3 | Group 4 |
|---|---|---|---|---|
| Before drug | 220 ± 4 | 222 ± 3 | 220 ± 3 | 221 ± 4 |
| administration | | | | |
| Day 1 | 223 ± 4 | 220 ± 2 | 206 ± 2 | 187 ± 2 |
| Day 7 | 221 ± 3 | 219 ± 3 | 190 ± 3 | 183 ± 1 |
| Day 14 | 221 ± 5 | 219 ± 4 | 190 ± 1 | 180 ± 1 |
| Day 28 | 219 ± 4 | 214 ± 2 | 189 ± 1 | 181 ± 2 |

### EXAMPLE 4

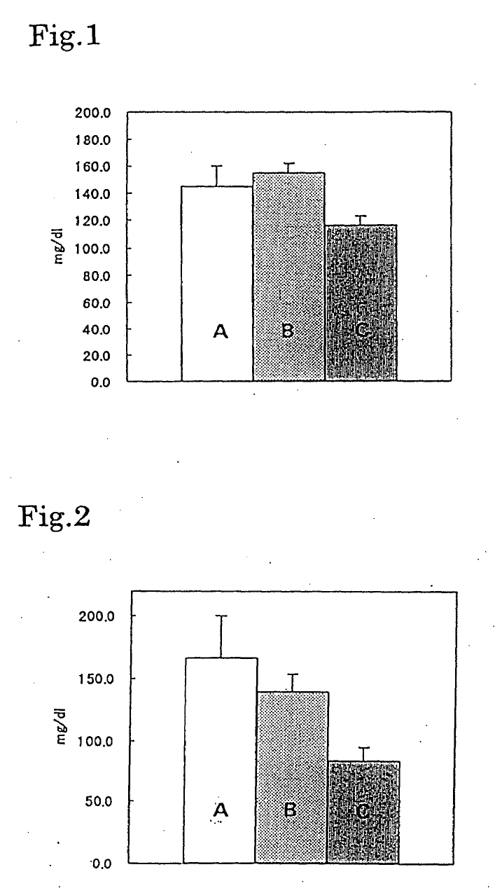

Male 6 weeks old SD rats were fed chow containing 1% cholesterol and 0.5% cholic acid, and pratosartan at a dose of 0.3 mg/kg or 3 mg/kg were administered once a day for 8 weeks simultaneously. Twenty-four hours after the last dose, blood samples were collected from abdominal aorta under ether anesthesia, and serum total cholesterol level and LDL cholesterol levels were determined. The results were shown in FIGS. 1 and 2. In the figures, A is the cholesterol value in the case of no pratosartan administration, B is the cholesterol value in the case of pratosartan administration at a dose of 0.3 mg/kg and B is the cholesterol value in the case of pratosartan administration at a dose of 3 mg/kg.

### EXAMPLE 5

107 patients with mild or moderately essential hypertension were administered a dose of from 40 to 160 mg/day every four weeks by degrees if the hypotensive action was insufficient. Serum cholesterol level before pratosartan administration was compared with that of the last day during pratosartan administration in each patient. Also, a part of the patients were co-administered pratosartan and conventionally known hypolipidemic drugs. General names of co-administered drugs, doses and number of patients were as follows. Namely, the list waspravastatin (10 mg/day, 3patients), simvastatin (5 mg/day, 3 patients), cerivastatin (0.15 mg/day, 2 patients), atorvastatin (5 mg/day, 1 patients), fenofibrate (150 mg/day, 1 patient) and probucol (500 mg/day, 1 patient). The results, when the patients were classified in the presence and absence of other co-administered drugs, are shown in Tables 6 and 7. The results in the presence of co-administered drugs, are shown in Table 6 and the results in the absence of the co-administered drug, are shown in FIG. 7. Further, data shown are mean±standard deviation.

TABLE 5

| | Serum cholesterol level (mg/dL) | | | |
|---|---|---|---|---|
| | | The last day | | |
| No. of | Before drug | during | | |
| patients | administration | administration | Difference | Paired t-test |
| 107 | 211.2 ± 42.6 | 199.6 ± 40.2 | −11.7 ± 29.7 | P < 0.0001 |
| | | | | (significantly) |

TABLE 6

| | Serum cholesterol level (mg/dL) | | | |
|---|---|---|---|---|
| | | The last day | | |
| No. of | Before drug | during | | |
| patients | administration | administration | Difference | Paired t-test |
| 12 | 250.25 ± 45.16 | 214.58 ± 37.31 | −35.67 ± 41.49 | P < 0.05 |
| | | | | (signifi- |
| | | | | cantly) |

TABLE 7

| | Serum cholesterol level (mg/dL) | | | |
|---|---|---|---|---|
| | | The last day | | |
| No. of | Before drug | during | | |
| patients | administration | administration | Difference | Paired t-test |
| 95 | 206.28 ± 39.81 | 197.66 ± 40.33 | −8.62 ± 26.60 | P < 0.01 |
| | | | | (signifi- |
| | | | | cantly) |

### FIELD OF INDUSTRIAL APPLICATION

Drugs in this invention are useful for treating hypertension combined with hyperuricemia and/or hypercholesterolemia, which are more frequent among the overlap of the risk factor in geriatric diseases.

What is claimed is:

1. A method of treating a patient having hypertension and at least one of serum hyperuricemia and hypercholesterolemia comprising the step of administering to the patient a pharmaceutically effective amount of 2-propyl-3-{[2′-(1H-tetrazole-5-yl)biphenyl-4-yl]methyl}-5,6,7, 8-tetrahydrocycloheptaimidazole-4-(3H)-one, or a pharmaceutically acceptable salt thereof.

2. The method of claim 1, wherein a diuretic is co-administered.

3. The method of claim 2, wherein the diuretic is one or more diuretics selected from the group consisting of a sulfonamide, a phenoxyacetic acid, a thiazide, triamterene, amiloride, spironolactone, potassium canrenoate and traxanox sodium.

4. The method of claim 2, wherein the diuretic is one or more thiazides selected from the group consisting of hydrochlorothiazide, methylclothiazide, benzylhydrocholorothiazide, trichloromethiazide, cyclopenthiazide, ethiazide, cyclothiazide, bendroflumethiazide and hydroflumethiazide.

5. A method of treating a patient having hypertension and hypercholesterolemia comprising the step of administering to the patient a pharmaceutically effective amount of 2-propyl-3-{[2′-(1H-tetrazole-5-yl)biphenyl-4-yl]methyl}-5,6,7, 8-tetrahydrocycloheptaimidazole-4-one, or a pharmaceutically acceptable salt thereof, and a hypolipidemic drug.

6. The method of claim 5, wherein the hypolipidemic drug is one or more hypolipidemic diuretics selected from the group consisting of a statin, a fibrate, a cholesterol absorption inhibitor, a cholesterol sequestrant and a cholesterol excretion enhancer.
